(19) United States
(12) Patent Application Publication
(10) Pub. No.: US 2014/0371532 A1
(43) Pub. Date: Dec. 18, 2014

(54) NESTED CANNULAS WITH GUIDED TOOLS

(71) Applicant: KONINKLIJKIE PHILIPS N.V., EINDHOVEN (NL)

(72) Inventor: Karen Irene Trovato, Putnam Valley, NY (US)

(21) Appl. No.: 14/369,703

(22) PCT Filed: Dec. 22, 2012
(86) PCT No.: PCT/IB2012/057662

§ 371 Date: Jun. 29, 2014

Related U.S. Application Data
(60) Provisional application No. 61/581,672, filed on Dec. 30, 2011.

(51) Int. Cl. A61B 17/34; A61B 17/28; A61B 17/221; A61B 17/3211

(52) U.S. Cl. CPC A61B 17/3423; A61B 17/3417; A61B 17/3211; A61B 17/28; A61B 17/221; A61B 2017/32113; A61B 2017/3443; A61B 2017/00991
USPC 600/114; 606/185; 606/167; 606/205; 606/113

(57) ABSTRACT

A medical instrument includes a guide (102) having an interlocking structure. A tool (104) is enclosed within the guide and has an interlocking feature configured to engage the interlocking structure of the guide. The tool has a stored position and a deployed position such that in transitioning between the stored position and the deployed position, motion of the tool relative to the guide is controlled in accordance with the interlocking structure.

This disclosure relates to medical devices and more particularly to nested cannulas or guides having a tool provided with one or more oriented mating components for guidance during an interventional procedure.

“Nested cannula” refers to a device constructed with nested, length-wise interlocking tubes, typically extended sequentially from largest to smallest. A commonly assigned pending application entitled “Nested Cannulae for Minimally Invasive Surgery”, International Publication No. WO 2009/156892, Nov. 10, 2010, which is incorporated herein by reference, in its entirety, discloses systems and methods for a nested cannula configuration to reach a target location within a particular anatomical region depending upon the requirements of the medical procedure. To employ a nested cannula by sequential deployment, the configuration of the tubes must be defined so that the path and the final pre-determined position of the distal tip may be achieved.

There are many minimally invasive tools including: loops, snares, scalpels, forceps, curved biopsy needle, sensors, imagers, etc. Minimally invasive tools often need to be oriented properly to be effective for their planned usage and to achieve their desired effect. If the tool is not oriented correctly, it may not provide correct readings or actions and can cause unwarranted damage. An Endo-Bronchial Ultrasound (EBUS) needle is an example of an imaging tool. The EBUS images tissue on one side of an airway. If the target is visualized, then a needle may be extended into the target. Naturally, if the needle is rotated incorrectly, the target may not be seen. Thus, a biopsy procedure cannot accomplish its objective until the EBUS is repositioned with the proper orientation. This takes time and expert hand-eye coordination.

In accordance with the present principles, a medical instrument includes a guide having an interlocking structure. A tool is enclosed within the guide and has an interlocking feature configured to engage the interlocking structure of the guide. The tool has a stored position and a deployed position such that in transitioning between the stored position and the deployed position, motion of the tool relative to the guide is controlled in accordance with the interlocking structure.

A medical instrument includes a nested cannula arrangement having a plurality of nested cannulas and an inner cannula having an interlocking structure formed on an interior portion thereof. A tool is enclosed within the inner cannula and has an interlocking feature configured to engage the interlocking structure of the inner cannula. A functional portion is affixed to a distal end portion of the tool and has a deployed position orientated in accordance with the interlocking feature relative to the interlocking structure such that upon deployment, motion of the functional portion relative to the inner cannula is controlled.

A system for performing a medical procedure includes a medical instrument including a guide having an interlocking structure and a tool enclosed within the guide and having an interlocking feature configured to engage the interlocking structure of the guide. The tool has a stored position and a deployed position such that in transitioning between the stored position and the deployed position, motion of the tool relative to the guide is controlled in accordance with the interlocking structure. A workstation is configured to monitor and control deployment of the medical instrument.

A method for deploying a medical instrument includes providing a medical instrument including a guide having an interlocking structure; and a tool enclosed within the guide and having an interlocking feature configured to engage the interlocking structure of the guide, the tool having a deployed position and a stored position such that in transitioning between the stored position and the deployed position, motion of the tool relative to the guide is controlled in accordance with the interlocking structure; planning a position and orientation of the medical instrument within a subject and deploying the tool from the planned position and orientation.

These and other objects, features and advantages of the present disclosure will become apparent from the following detailed description of illustrative embodiments thereof, which is to be read in connection with the accompanying drawings.

This disclosure will present in detail the following description of preferred embodiments with reference to the following figures wherein:

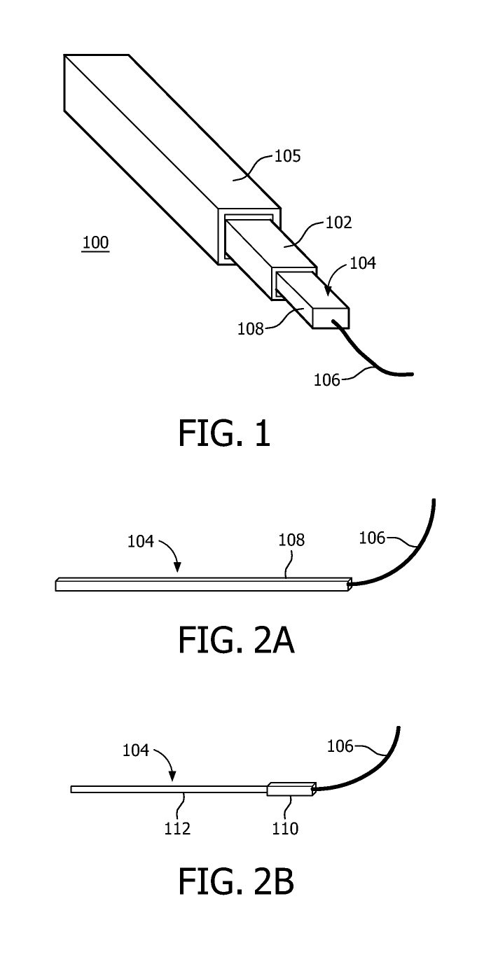

FIG. 1 is a perspective view of a nested cannula having a tool guided by interlocking structures in the cannula in accordance with one illustrative embodiment;

FIG. 2A is a side perspective view of a tool with an interlocking feature along its entire length, which is guided by interlocking structures in the cannula of FIG. 1 in accordance with one illustrative embodiment;

FIG. 2B is a side perspective view of a tool with an interlocking feature along a portion of its entire length, which is guided by interlocking structures in the cannula in accordance with another illustrative embodiment;

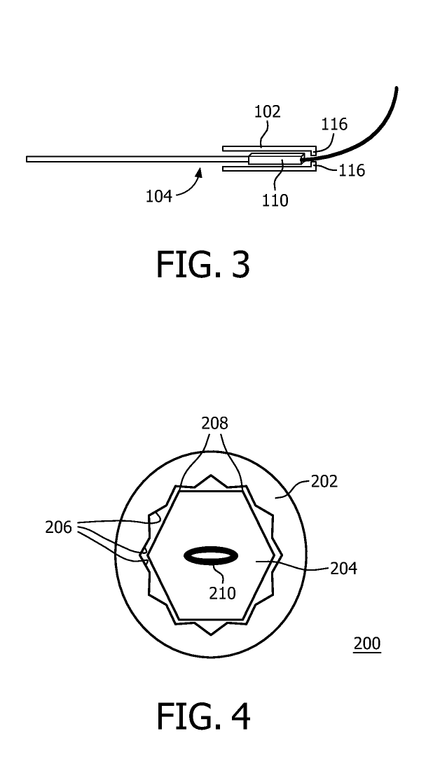

FIG. 3 is a cross-sectional view showing a tool having a stop formed by the cannula in accordance with the illustrative embodiment;

FIG. 4 is a cross-sectional view of a cannula having interlocking features that include ridges or contours and having a tool disposed therein with corresponding interlocking features that can be selectively keyed to provide an particular angular relation between the tool and its guide in accordance with another illustrative embodiment;

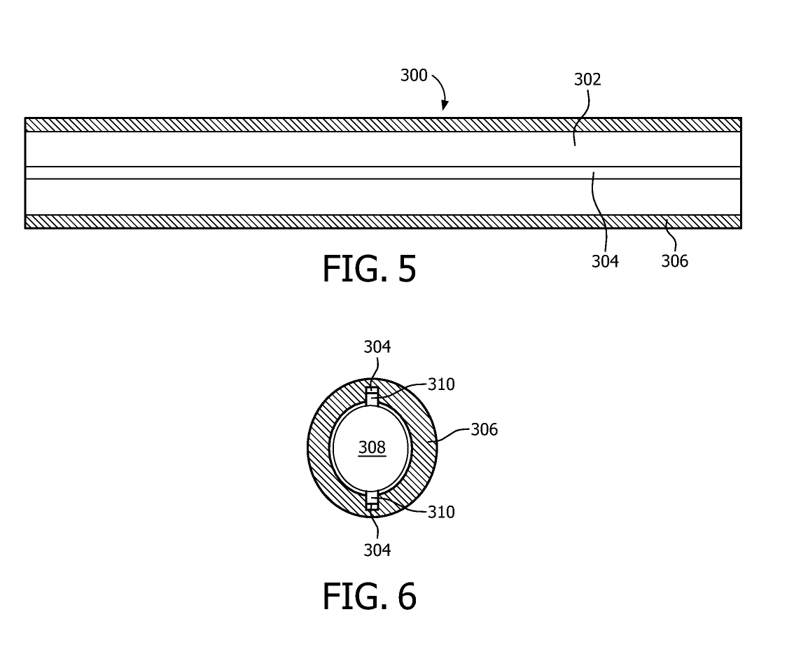

FIG. 5 is a cross-sectional view of a section taken longitudinally of a guide or cannula in accordance with one illustrative embodiment;

FIG. 6 shows a cross-sectional view taken perpendicular to the longitudinal direction of the guide of FIG. 5 with a tool shaft or bead provided therein in accordance with an illustrative embodiment;

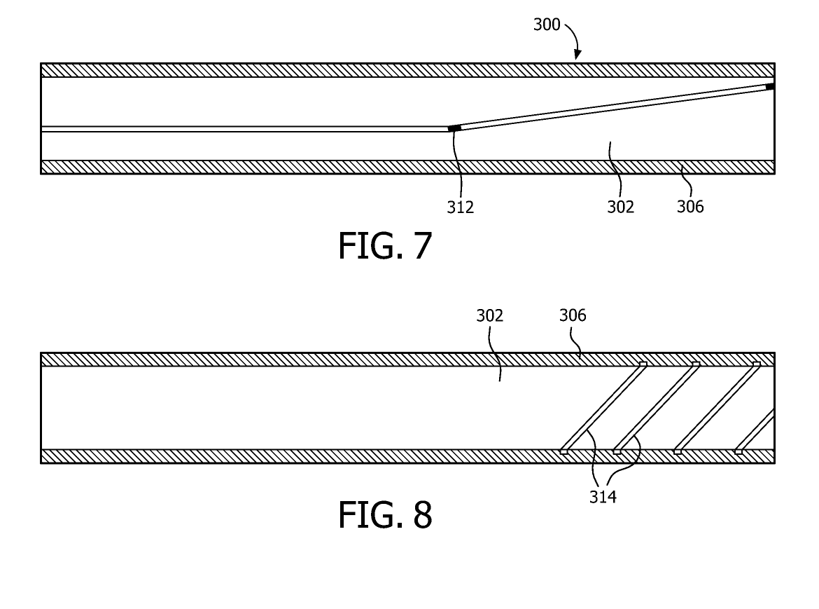

FIG. 7 is a cross-sectional view of a section taken longitudinally of a guide or cannula with a groove for partially twisting a tool in accordance with another illustrative embodiment;

FIG. 8 is a cross-sectional view of a section taken longitudinally of a guide or cannula with a groove for rotating a tool in accordance with another illustrative embodiment;

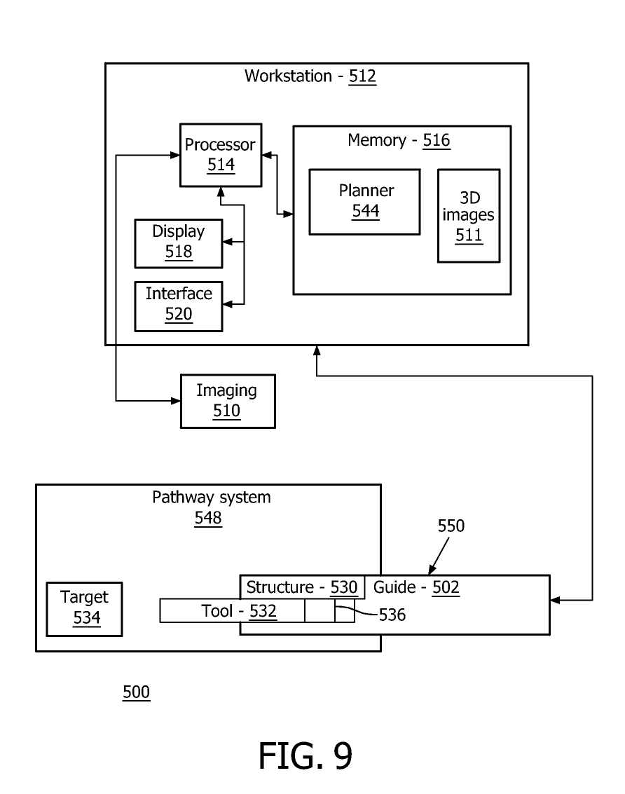

FIG. 9 is a block diagram showing a system for performing a medical procedure in accordance with the present principles; and

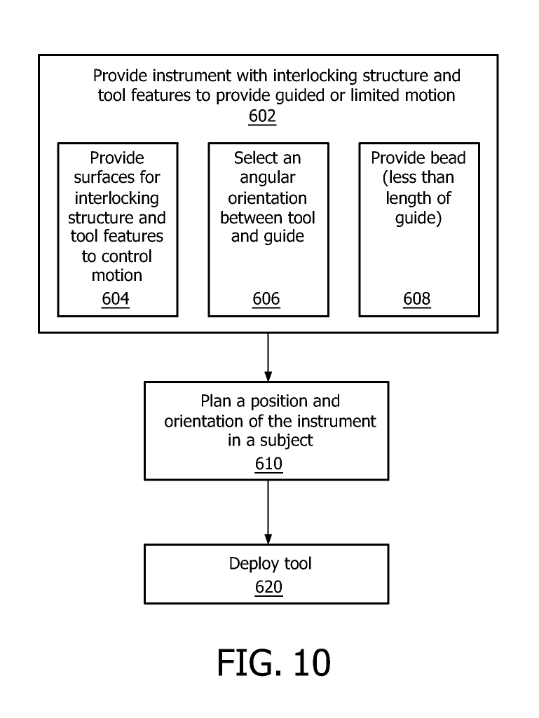

FIG. 10 is a flow diagram showing steps for performing a medical procedure in accordance with the present principles.

The present embodiments provide a cannula, nested cannula, channels or other guides that are configured to deliver a tool or tools therein for carrying out a procedure. In accordance with the present principles, an innermost cannula has a component disposed therein having a functional portion or a tool attached to its distal end portion. The innermost component, which may also be referred to generally as a tool has a geometric relationship with its nearest neighboring tube. This relationship permits the innermost component to longitudinally travel down the nearest neighboring tube without rotation in one embodiment and may be rotated a controlled amount in another embodiment. In this way, the orientation of the tool (innermost component) is controlled to enable proper deployment.

In another embodiment, a functional portion of a tool is delivered by a push rod or other instrument, which permits the tool to longitudinally travel down the nearest neighboring tube with or without rotation by providing a bead or section adjacent to the functional portion. The bead is configured to have a geometric relationship with its nearest neighboring tube. The cannulas, guides and/or tools are configured with features to mechanically control, orient or sustain motion of the tools. The tools are held in a steady orientation as the tools are extended by having an interlocking feature that matches an interlocking shape of a surrounding tube of the guide or cannula. This permits the tool to resist twisting or other displacement as the tool crosses anatomical boundaries, interstitial regions, etc. within the cannula to a target.

In one embodiment, a cannula is configured to receive a keyed tool. The keyed tool includes one or more flats, protrusions, grooves, teeth, keys, etc. along its length, which engage features within the cannula to guide the tools out from the cannula with a particular motion. In another embodiment, the keys on the tool prevent rotation of the tool relative to the cannula during the usage of the tool, e.g., during a procedure.

It should be understood that the present invention will be described in terms of medical instruments; however, the teachings of the present invention are much broader and are applicable to any instruments employed in repairing or analyzing complex biological or mechanical systems. In particular, the present principles are applicable to internal investigations and procedures for biological systems, procedures in all areas of the body such as the lungs, gastro-intestinal tract, excretory organs, brain, blood vessels, etc. The elements depicted in the FIGS. may be implemented in various combinations of hardware and may include software guidance systems and provide functions which may be combined in a single element or multiple elements.

Referring now to the drawings in which like numerals represent the same or similar elements and initially to FIG. 1, a cross-sectional view of a device 100 shows a guide 102 and a tool 104 therein in accordance with one embodiment. The guide 102 may include, e.g., a cannula, a channel within a device (e.g., in an endoscope), a nested cannula, or any other guide. FIG. 1 shows a nested cannula arrangement where guide 102 is nested within another guide or tube 105. It should be understood that the nested cannula arrangement may include more than two cannulas. The tool 104 may include a functional portion 106 that may include, e.g., a loop, a snare, a scalpel, a needle, forceps, imaging probe or any other device employed during a procedure that is adapted to pass through a tube or cannula. FIG. 1 illustratively shows a needle 106 affixed to an end portion of the tool 104. The present embodiments provide a working relationship between the guide 102 and the tool 104 such that when the tool 104 is positioned in the guide 102, an interlocking structure or relationship limits or permits motion of the tool 104 relative to the guide 102.

In the embodiment depicted in FIG. 1, the tool 104 includes a rectangular cross-section shaft 108 (or bead 110, FIG. 2B) that fits within the guide or outer tube 102. In this case, the geometric relationship between the tool 104 and the guide 102 provides for centering and orienting the deployment of the needle 106. In addition, the geometry of the tool 104 provides a torque stop function to prevent rotation of the tool 104 with respect to its guide 102. Shaft 108 slides longitudinally along the interior of guide 102 but may have its longitudinal reach limited as well. Here, the interlocking structure of the guide 102 is its cornered rectangular shape, and the shaft 108 includes a corresponding interlocking feature, e.g., its rectangular fitting shape.

Referring to FIGS. 2A and 2B with continued reference to FIG. 1, two illustrative embodiments for tool 104 are shown. In FIG. 2A, tool 104 includes a tube or solid shaft 108 (as an example of an interlocking structure) that is configured to fit inside guide 102. Shaft 108 may have a substantially uniform cross-section and have functional portion 106 (e.g., a needle or other device) attached on a distal end portion thereof. The cross-section of shaft 108 corresponds with the inner surfaces of the guide 102 to resist rotation of the needle 106 during its use. The shaft 108 and the inner surfaces of guide 102, in this example, include mating flat surfaces of the rectilinear shaped cross-sections to provide one illustrative form of an interlocking structure between the guide 102 and the shaft 108 of tool 104. The needle 106 may be employed to penetrate a boundary, such as, e.g., a lung wall (or other tissue) to biopsy tissue, etc. Since the needle 106 needs to penetrate the lung wall, force applied to the tool 104 to result in needle penetration would result in a reaction force on the tool 104 from the needle 106. This reaction force would normally result in a twisting of the tool 104. However, due to the relationship between the tool 104 and the cannula structure (guide 102) namely the flat surfaces in this case, twisting is resisted resulting in a more accurate and controllable needle deployment. Furthermore, by the mere deployment of the tool 104 from a nested cannula, the tool is preferably pre-oriented in its correct position.

In FIG. 2B, a bead 110 (which may include a portion of the shaft 108) is provided adjacent to the functional portion 106 (e.g., needle) for tool 104. In this example, the needle 106 is affixed to the bead 110, although other configurations are contemplated. The bead 110 may include a gear shape having one or more teeth that interlock with corresponding shapes on the interior of a surrounding tube. The needle 106 with the bead 110 may be deployed using a push-rod 112 or similar device (e.g., a wire, etc.). The push-rod 112 may be detachable from the bead 110, and the bead 110 would preferably have an attached string (not shown). The string can remain in place with the bead 110 and tool 106, yet be long enough to provide a mechanism to remove the bead and tool by pulling it from outside the body. The bead 110 includes similar features corresponding to the interlocking structure of internal surfaces of the guide 102 and includes a same cross-section as the shaft 108, as described above, but the bead 110 extends only a short longitudinal distance. The shorter longitudinal distance reduces friction between the inner surfaces of the guide 102. However, twisting resistance and centered positioning of the needle 106 is maintained due to the interlocking structure between the guide 102 and the bead 110. The length of the bead 110 is preferably shorter than the length of the guide 102.

Referring to FIG. 3, it should be understood that the interlocking structure or arrangement between the interlocking shape of bead 110 and the guide 102 may include a stopping surface 116 to prevent distal advancement of the bead 110 out of the guide 102. This structure is also useful with the shaft 108. Bead 110 may engage stopping surface 116 to prevent distal motion of the tool 104 to ensure that the tool 104 can easily be backed out after use. Other configurations are also contemplated such as providing a string as an additional safety to aid in the removal of the tool, e.g., providing a string to pull the bead 110 back into the guide if the guide length is exceeded during a procedure. If an interlocking object or bead needs to be pulled back into a parent housing (cannula) appropriate configuration is preferred, e.g., tapered edges, rounded or conical back-end features, etc.

Referring to FIG. 4, an end view of a nested cannula set 200 in accordance with another embodiment is illustratively shown. Set 200 employs two or more telescoping components 202 and 204 with each having a pre-set interlocking shape and a pre-set curvature. For the outermost tube 202 of the set 200, the pre-set interlocking shape is relevant for its inner surface, and for the innermost component 204 of the set 200, the pre-set interlocking shape is relevant for the outer surface. For any intermediate tube of the set, the pre-set interlocking shape is relevant for both the internal and outer surfaces of such tube.

The interlocking shape of each component is any shape that interlocks an inner component to an outer tube whenever the inner component is nested within the outer tube whereby any individual rotation about a gap therebetween by the inner component is limited by the outer tube and any individual rotation about the gap therebetween by the outer tube is limited by the inner component. Such interlocking shapes for the components include, but are not limited to, a polygonal interlocking shape, a non-circular closed curve interlocking shape (e.g., oval), a polygonal-closed curve interlocking shape, a keyway interlocking shape, etc. Another variety of interlocking shapes relies on non-scaled versions of a single shape, for example, a rectangle or triangle interlocked within a hexagon or other polygon. One example may include finer ridges or contours inside and less frequent ridges or contours outside or vice versa (e.g., the inside surface is not just a slightly smaller scale of the outside surface).

In one illustrative embodiment, as depicted in FIG. 4, multiple ridges 206 are provided on an inner surface of tube 202. Corners 208 of a shape of innermost component 204 engage these ridges 206 to provide rotational resistance as described above. FIG. 4 shows a hexagonal shaped innermost component 204 but could also be a triangle, square, or any other polygon or ridged shaped cross-section. An instrument 210 (e.g., a needle, etc.) would be affixed to the innermost component 204. During deployment, the interlocking structure between the ridges 206 of tube 202 and the corners 208 of component 204 provide rotation resistance. In addition, the orientation of the instrument can be planned and adjusted for deployment by selecting an angular position of the component 204 with respect to the tube 202. Since there is no relative rotation between components 202 and 204, a desired deployment of the instrument 210 connected with the component 204 can be made. Using the example of a needle, the needle may have a desired orientation which can be pre-determined and provided in advance. The component 204 has its connected instrument 210 set to its angular position relative to the corners/ridges provided on components 202 and 204. This enables the component 204 to be set at one of many different angles for deployment. One particularly useful embodiment includes nested hexagonal shapes.

Other embodiments may also be designed and employed in accordance with the present principles. Referring to FIGS. 5 and 6, FIG. 5 shows a longitudinal cross-sectional view of a guide or cannula 300 and FIG. 6 shows a cross-sectional view of the guide 300 perpendicular to the longitudinal direction with a tool shaft or bead 308 provided therein. In one embodiment, the cannula or guide 300 includes a groove or grooves 304 in its side wall 306 on an interior surface 302. In this case, the grooves 304 comprise the interlocking structure of the guide 300 and protrusions 310 on the bead or shaft 308 of a tool include the interlocking features. The protrusions 310 fit in the grooves 304 and may provide rotation during deployment. Note that the protrusions 310 and grooves 304 may be reversed such that the grooves are formed in the tool 308 and the protrusions are formed on the interior surface 302 of the guide 300. The grooves 304 and/or protrusions 310 need not extend over the entire longitudinal distance of the guide 300. Other interlocking configurations are also contemplated.

The grooves 304 or protrusions 310 may be configured to provide different motions or actions for the tool 308. Referring to FIG. 7, a groove (or protrusion) 312 veers off on an angle to cause a twist in the tool 308 as it exits the cannula or guide 300. Note that the amount or twist is provided to control the placement of the tool in a beneficial or predictable way. Referring to FIG. 8, grooves 314 are provided to cause a rotation or multiple twists of the tool 308 upon exit. A lead-in portion of the grooves 314 is not shown. Multiple grooves may be provided for multiple protrusions (e.g., on opposing sides of the tool 308) on the tool to provide stability. In one embodiment, four grooves 314 are employed and configured to each receive a corner of square shaped bead (110) or flexible shaft (108) to rotate the bead or flexible shaft. Rotation is a difficult motion to provide in this case as the grooves need to be appropriately dimensioned, and/or the shaft that carries the bead needs to be sufficiently flexible. It is preferable that the bead is round with protrusions that may be like gear teeth if there is a desired rotation motion. The gear teeth need not be the full length of the tube or bead in this case. In another example, if the interlock for turning is a hex shape or the like, then the inner component needs to be flexible enough to twist at the rotating end.

Referring to FIG. 9, a system 500 for designing and using cannulas in accordance with the present principles is illustratively shown. The functions of the various elements shown in FIGS. 9 and 10 can be provided through the use of dedicated hardware as well as hardware capable of executing software in association with appropriate software. When provided by a processor, the functions can be provided by a single dedicated processor, by a single shared processor, or by a plurality of individual processors, some of which can be shared. Moreover, explicit use of the term “processor” or “controller” should not be construed to refer exclusively to hardware capable of executing software, and can implicitly include, without limitation, digital signal processor (“DSP”) hardware, read-only memory (“ROM”) for storing software, random access memory (“RAM”), non-volatile storage, etc.

Moreover, all statements herein reciting principles, aspects, and embodiments of the invention, as well as specific examples thereof, are intended to encompass both structural and functional equivalents thereof. Additionally, it is intended that such equivalents include both currently known equivalents as well as equivalents developed in the future (i.e., any elements developed that perform the same function, regardless of structure). Thus, for example, it will be appreciated by those skilled in the art that the block diagrams presented herein represent conceptual views of illustrative system components and/or circuitry embodying the principles of the invention. Similarly, it will be appreciated that any flow charts, flow diagrams and the like represent various processes which may be substantially represented in computer readable storage media and so executed by a computer or processor, whether or not such computer or processor is explicitly shown.

Furthermore, embodiments of the present invention can take the form of a computer program product accessible from a computer-usable or computer-readable storage medium providing program code for use by or in connection with a computer or any instruction execution system. For the purposes of this description, a computer-usable or computer readable storage medium can be any apparatus that may include, store, communicate, propagate, or transport the program for use by or in connection with the instruction execution system, apparatus, or device. The medium can be an electronic, magnetic, optical, electromagnetic, infrared, or semiconductor system (or apparatus or device) or a propagation medium. Examples of a computer-readable medium include a semiconductor or solid state memory, magnetic tape, a removable computer diskette, a random access memory (RAM), a read-only memory (ROM), a rigid magnetic disk and an optical disk. Current examples of optical disks include compact disk-read only memory (CD-ROM), compact disk-read/write (CD-R/W) and DVD.

Cannulas, nested cannulas or guides as described herein may be designed to be task specific devices. Once correctly guided and positioned in a patient, these cannulas, nested cannulas, or guides are deployed for one or more specific tasks. System 500 may include a workstation or console 512 from which a procedure is supervised and managed.

Workstation 512 preferably includes one or more processors 514 and memory 516 for storing programs and applications. Memory 516 may store modules or software tools configured to interpret feedback signals or provide guidance and control of tools employed during a procedure. A planner 544 may be employed to design an instrument 550 (e.g., device 100 of FIG. 1), such as a nested cannula system or a guide system, by providing arcs, lengths and orientations of cannula segments of the instrument 550 in a patient (e.g., an anatomical system) or a pathway system 548 (e.g., a pipe system, a wiring conduit, etc.).

The instrument 550 is preferably elongated and includes at least one guide or outer cannula 502 for deploying a tool 532. The guide 502 may include e.g., a cannula, a nested cannula, a tube or other guide. The tool 532 (e.g., functional portion 106, FIG. 1) may include forceps, a loop, a trocar, a wire, a scope, a probe, an electrode, a needle, a scalpel, a probe, a balloon, ablation device (RFA, radiation, chemo, cryo), imaging device (fiber-optic, CCD), sensing device (temperature, pressure) or other medical component. Workstation 512 may include a display 518 for viewing internal images of the subject 548.

In one embodiment, a tracking system monitors progress of the deployment of the instrument 550, e.g., an imaging system 510, such as a C-arm fluoroscopy system, whereby the images received are compared to original computed tomography (CT) or other pre-operative images of a target to validate reaching a final location. The imaging system 510 may include, e.g., a magnetic resonance imaging (MRI) system, a fluoroscopy system, a computed tomography (CT) system, ultrasound (US), etc. Display 518 may also permit a user to interact with the workstation 512 and its components and functions. This is further facilitated by an interface 520 which may include a keyboard, mouse, a joystick or any other peripheral or control to permit user interaction with the workstation 512.

Imaging system 510 may be provided for collecting pre-operative imaging data or real-time inter-operative imaging data. The pre-operative imaging may be performed at another facility, location, etc. in advance of any procedure. These images 511 may be stored in memory 516, and may include pre-operative 3D image volumes of a patient or pathway system. Images 511 are preferably employed in designing the instrument 550, e.g., determining its dimensions and orientations for each nested portion for surgery and/or its deployment.

In a particularly useful embodiment, instrument 550 is employed to remove, examine, treat, etc. a target 534. The target 534 may include a lesion, tumor, injury site, object, etc. During a procedure, the instrument 550 is deployed to reach the target 534. The tool 532, its interlocking shapes or features 536, the guide 502 and its interlocking structure 530 are designed and configured in advance of a procedure and may be designed based on input from the images 511. For example, the planner 544 employs the image and target data available for a specific patients' anatomy to plan the procedure and design the tool 532, etc. to be proportioned with the other nested components (e.g., guide 502) so that it reaches the intended target 534. Also, the angular position of the tool 532 needs to be selected using the interlocking features 536 and the interlocking structure 530 so that an oriented tool that faces toward a region of interest is achieved to orient the tool face precisely. A patient-specific device 550 can be simulated, approved, manufactured and delivered in a short period of time.

As described above, the guide 502 may include interlocking structures 530 that interact with interlocking shapes or features 536 of the tool 532 (depicted for illustratively in the FIG. 9). In this way, the motion of the tool during deployment is beneficially controlled or limited. During a procedure, the instrument or device 550 is deployed to a location, say in a lung. A position and orientation of the instrument 550 is determined based upon its design. An angular position of the tool 532 may be selected to give a desired orientation/predetermined position in accordance with the plan or design the instrument 550. The tool 532 is then deployed from the guide 502 to perform its intended purpose. The motion, displacement, rotation, etc. of the tool 532 is controlled based upon the interlocking structures 530 and its interaction with the shape or features 536 of the tool 532. It should be understood that multiple nested stages may be deployed in the same way and may include interlocking structures and corresponding interlocking shapes. The tool 532 with the interlocking components or shapes 536 supports the orientation of the tool 532 as it extends through at least one enclosing straight or curved guide 502.

Referring to FIG. 10, a method for deploying a nested medical instrument is illustratively shown. In block 602, a medical instrument, preferably, a nested cannula, is provided which includes a guide having an interlocking structure. A tool is enclosed within the guide and has an interlocking feature configured to engage the interlocking structure of the guide. The tool has a deployed position and a stored position such that in transitioning between the stored position and the deployed position, motion of the tool relative to the guide is controlled in accordance with the interlocking structure.

In block 604, the interlocking structure may include one or more flat surfaces, curved surfaces, protrusions, grooves, combinations thereof, etc., and the interlocking feature may include a corresponding feature(s) such that, when in the deployed position, rotation and translation of the tool are permitted or resisted in a controlled manner. In block 606, the interlocking structure may include a plurality of angular positions, and the interlocking feature includes a surface that engages the interlocking structure to provide a selection of one fixed angular position of the tool relative to the guide. In block 608, the interlocking feature may include a bead that extends less than a length of the guide.

In block 610, a position and orientation of the medical instrument is planned within a subject. This may include consulting preoperative images, which results in the design of the cannula structure. This may be performed using a planner tool.

The nested cannula is deployed first into a patient or system. Then, in block 620, the tool is deployed from the planned position and orientation from within the nested cannula during a procedure.

In interpreting the appended claims, it should be understood that:
a) the word “comprising” does not exclude the presence of other elements or acts than those listed in a given claim;
b) the word “a” or “an” preceding an element does not exclude the presence of a plurality of such elements;
c) any reference signs in the claims do not limit their scope;
d) several “means” may be represented by the same item or hardware or software implemented structure or function; and
e) no specific sequence of acts is intended to be required unless specifically indicated.

Having described preferred embodiments for systems, devices and methods for nested cannulas with guided tools (which are intended to be illustrative and not limiting), it is noted that modifications and variations can be made by persons skilled in the art in light of the above teachings. It is therefore to be understood that changes may be made in the particular embodiments of the disclosure disclosed which are within the scope of the embodiments disclosed herein as outlined by the appended claims. Having thus described the details and particularity required by the patent laws, what is claimed and desired protected by Letters Patent is set forth in the appended claims.

1. A medical instrument, comprising:
a guide (102) having an interlocking structure; and
a tool (104) comprising a component having a functional portion extendable from a distal end portion of the tool, said tool is enclosed within the guide and having an interlocking feature configured to engage the interlocking structure of the guide;
the tool having a stored position and a deployed position wherein the functional portion extends beyond the interlocking structure of the guide such that in transitioning between the stored position and the deployed position, motion of the tool relative to the guide is controlled in accordance with the interlocking structure.

2. The instrument as recited in claim 1, wherein the interlocking structure (206) includes one or more of a flat, a key, a groove, a protrusion, a corner and a surface, and the interlocking feature (208) includes at least one corresponding surface on a rigid portion such that, when in the deployed position, rotation of the tool is resisted.

3. The instrument as recited in claim 1, wherein the interlocking structure (206) includes a plurality of angular positions and the interlocking feature (208) includes a surface that engages the interlocking structure to provide a selection of one fixed angular position of the tool relative to the guide.

4. The instrument as recited in claim 1, wherein the interlocking structure (206) includes contours or ridges and the interlocking feature (208) includes one or more corners.

5. The instrument as recited in claim 1, wherein the guide (102) includes a tube and the interlocking structure slidably engages an interior portion of the tube.

6. The instrument as recited in claim 5, wherein the tube is nested inside another tube (105).

7. The instrument as recited in claim 1, wherein the functional portion (106) of the tool includes one or more of a needle, a scalpel, a forceps, a loop, a probe and a snare.

8. The instrument as recited in claim 1, wherein the interlocking feature includes a bead (110) that extends less than a length of the guide.

9. A medical instrument, comprising:
a nested cannula arrangement (100) having a plurality of nested cannulas and an inner cannula (102) having an interlocking structure formed on an interior portion thereof;
a tool (104) comprising a component having a functional portion extendable from a distal end portion of the tool, said tool is enclosed within the inner cannula and having an interlocking feature configured to engage the interlocking structure of the inner cannula; and
the functional portion (106) of the tool having a deployed position wherein the functional portion extends beyond the interlocking structure of the inner cannula, said deployed position orientated in accordance with the interlocking feature relative to the interlocking structure such that upon deployment, motion of the functional portion relative to the inner cannula is controlled.

10. The instrument as recited in claim 9, wherein the interlocking structure (206) includes one or more of a flat, a key, a groove, a protrusion, a corner and a surface, and the interlocking feature (208) includes at least one corresponding surface on a rigid portion such that, when in the deployed position, rotation of the tool is resisted.

11. The instrument as recited in claim 9, wherein the interlocking structure (206) includes a plurality of angular positions and the interlocking feature (208) includes a surface that engages the interlocking structure to provide a selection of one fixed angular position of the tool relative to the guide.

12. The instrument as recited in claim 9, wherein the interlocking structure (206) includes contours or ridges and the interlocking feature (208) includes one or more corners.

13. The instrument as recited in claim 9, wherein the functional portion (106) includes one or more of a needle, a scalpel, a forceps, a loop, a probe and a snare.

14. The instrument as recited in claim 9, wherein the interlocking feature includes a bead (110) that extends less than a length of the guide.

15. A system for performing a medical procedure, comprising:
a medical instrument including:
a guide (502) having an interlocking structure (530); and
a tool (532) comprising a component having a functional portion extendable from a distal end portion of the tool, said tool is enclosed within the guide and having an interlocking feature (536) configured to engage the interlocking structure of the guide, the tool having a stored position and a deployed position wherein the functional portion extends beyond the interlocking structure of the guide such that in transitioning between the stored position and the deployed position, motion of the tool relative to the guide is controlled in accordance with the interlocking structure; and
a workstation (512) configured to monitor and control deployment of the medical instrument.

16.-24. (canceled)
